(19) United States
(12) Patent Application Publication
(10) Pub. No.: US 2025/0109686 A1
(43) Pub. Date: Apr. 3, 2025

(54) METHOD FOR ASSESSING WELLBORE GAS MEASUREMENTS

(71) Applicant: Schlumberger Technology Corporation, Sugar Land, TX (US)

(72) Inventors: Ivan Fornasier, Paris (FR); Dariusz Strapoc, Le Plessis-Robinson (FR); Pierrick Ferrando, Magny-Le-Hongre (FR); Giuseppe Matteo Cera, Paris (FR)

(21) Appl. No.: 18/593,071

(22) Filed: Mar. 1, 2024

(30) Foreign Application Priority Data
Sep. 28, 2023 (EP) 23306630.7

(51) Int. Cl. E21B 49/00; E21B 21/06; E21B 45/00; G01N 33/00

(52) U.S. Cl. CPC E21B 49/005; E21B 21/067; E21B 45/00; G01N 33/0027

(57) ABSTRACT

A method for assessing wellbore gas measurements includes determining drilling and environmental conditions corresponding to the measurements and computing an interpretation confidence index for the measurement from selected ones of the drilling and environmental conditions. The interpretation confidence index provides an assessment of the how well the measured composition reflects an actual reservoir gas composition.

### CROSS REFERENCE TO RELATED APPLICATIONS

This application claims priority to and the benefit of European Application No. 23306630.7, filed on Sep. 28, 2023, the entirety of which is incorporated herein by reference.

### BACKGROUND

When drilling a well for the production of hydrocarbons, drilling fluid is often circulated through the well for a number of purposes. For example, drilling fluid is commonly intended to provide pressure to the subterranean formation, cool and lubricate the drill bit, flush cuttings away from the drill bit and carry them to the surface, and provide hydraulic power to various downhole tools. Drilling fluids also commonly carry formation fluids and dissolved formation gasses to the surface. Such gasses may be liberated by the drill bit as it cuts the formation and may include various alkane gasses such as methane (C1), ethane (C2), propane (C3), butane (C4), pentane (C5), and the like, as well as artificially generated alkenes, alcohols and other polar contaminants originating from drilling operation-driven degradation of the drilling fluids.

The liberated gases are commonly evaluated at the surface while drilling (e.g., via gas chromatography). Such measurements may provide valuable information to a mud logger and may provide information about the maturity and nature of hydrocarbons in the reservoir, compartmentalization of intervals in the reservoir being drilled, and oil quality, as well as information regarding production zones, lithology changes, history of reservoir accumulation, seal effectiveness, and environmental impact of the drilling operation.

While such gas measurements may provide valuable insight about the contents of the reservoir, the gas concentrations measured at the surface can sometimes be different (even significantly different) than the actual reservoir gas composition. There is a need in the industry to assess how well gas composition measurements made at the surface reflect the actual reservoir gas composition.

### BRIEF DESCRIPTION OF THE DRAWINGS

For a more complete understanding of the disclosed subject matter, and advantages thereof, reference is now made to the following descriptions taken in conjunction with the accompanying drawings, in which:

FIG. 1 depicts an example drilling rig including a disclosed system for making and assessing gas composition measurements.

FIG. 2 depicts another embodiment of a surface system for making and assessing gas composition measurements.

FIGS. 3A and 3B (collectively FIG. 3) depict flow charts of example methods for making and assessing gas composition measurements.

FIGS. 4A, 4B, 4C, 4D, and 4E (collectively FIG. 4), depict example look-up tables for assigning coefficients from which an interpretation confidence index may be determined.

FIG. 5 depicts a graphical representation of coefficients for a plurality of environmental and measurement conditions.

### DETAILED DESCRIPTION

Embodiments of this disclosure include methods and systems for making and assessing drilling fluid gas composition measurements. In one example embodiment, a disclosed method includes measuring a composition of a gas sample obtained during a mud logging operation. The gas sample includes at least selected alkane gases. The method further includes determining drilling and environmental conditions corresponding to the measurement and computing an interpretation confidence index for the measured composition from selected ones of the drilling and environmental conditions. The interpretation confidence index is intended to provide an assessment of the how well the measured composition reflects an actual reservoir gas composition.

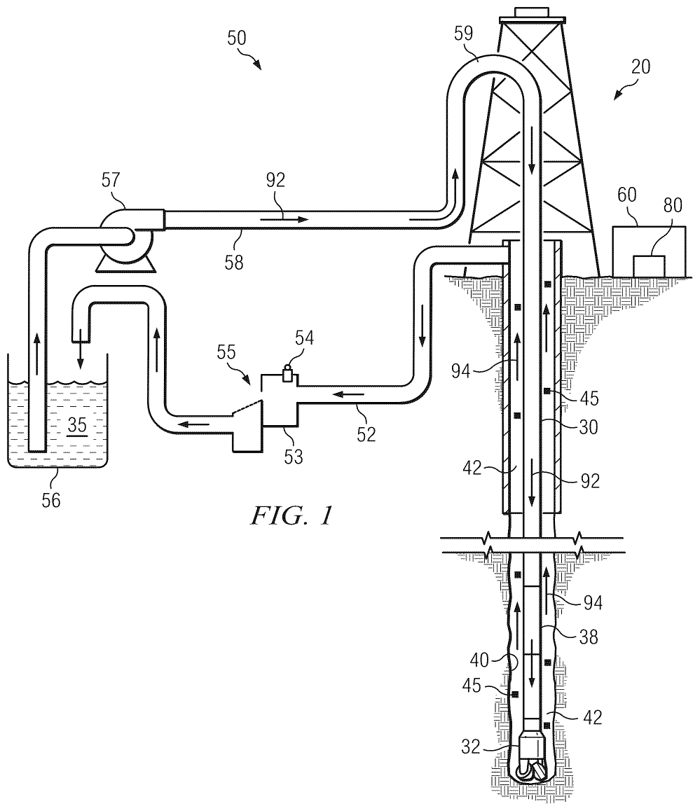

FIG. 1 depicts an example drilling rig 20 including a system 80 for making and assessing drilling fluid gas composition measurements. The drilling rig 20 may be positioned over a subterranean formation (not shown). The rig 20 may include, for example, a derrick and a hoisting apparatus (also not shown) for raising and lowering a drill string 30, which, as shown, extends into wellbore 40 and includes, for example, a drill bit 32 and one or more downhole measurement tools 38 (e.g., a logging while drilling tool or a measurement while drilling tool) in a bottom hole assembly (BHA) above the bit 32. Suitable drilling systems, for example, including drilling, steering, logging, and other downhole tools are well known in the art.

Drilling rig 20 further includes a surface system 50 for controlling the flow of drilling fluid used on the rig (e.g., used in drilling the wellbore 40). In the example rig depicted, drilling fluid 35 may be pumped downhole (as depicted at 92), for example, via a conventional mud pump 57. The drilling fluid 35 may be pumped, for example, through a standpipe 58 and mud hose 59 in route to the drill string 30. The drilling fluid 35 typically emerges from the drill string 30 at or near the drill bit 32 and creates an upward flow 94 of mud through the wellbore annulus 42 (the annular space between the drill string and the wellbore wall). The drilling fluid 35 then flows through a return conduit 52 to a mud pit system 56 where may be recirculated. It will be appreciated that the terms drilling fluid and mud are used synonymously herein.

The circulating drilling fluid 35 is intended to perform many functions during a drilling operation, one of which is to carrying drill cuttings 45 to the surface (in upward flow 94). The drill cuttings 45 are commonly removed from the returning mud via a shale shaker 55 (or other similar solids control equipment) in the return conduit (e.g., immediately upstream of the mud pits 56). Gases that are released or generated during drilling may also be carried to the surface in the circulating drilling fluid. These gasses, which may be dissolved in the mud or in the form of bubbles, are commonly removed from the drilling fluid, for example, via one or more degassers 54 located in or near a header tank 53 that is immediately upstream of the shale shaker 55 in the example depiction. The drill cuttings 45 and the extracted gases are commonly examined at the surface to assist the drilling operation and to evaluate the formation layers and the reservoir though which the wellbore is drilled.

As is known to those of ordinary skill in the art, formation gas may be released into the wellbore 40 via the drilling process (e.g., crushing the formation rock by the mechanical action of the drill bit) and may also migrate into the wellbore 40, for example, via fractures in the formation rock. The drilling process may also generate gases, for example, via drill bit metamorphism (DBM). Once in the wellbore, the gases may be transported to the surface via the drilling fluid (in the upwardly flowing fluid 94). The gases may be in solution (dissolved) in the drilling fluid and/or in the form of bubbles and may be sampled in the surface system, for example, via the one or more drilling fluid degassers 54 and/or a head space gas probe. The disclosed embodiments are not necessarily limited in regards to how the gas is sampled.

With further reference to FIG. 1, drilling rig 20 may further include a testing facility 60 (e.g., a mud logging system or a laboratory trailer including one or more instruments suitable for making various measurements of sampled gases in the drilling fluid). In the depicted embodiment, the testing facility 60 has instrumentation (such as a gas chromatography apparatus) and is configured to measure the formation gas composition. The testing facility 60 may, of course, include numerous other testing instruments known to those of ordinary skill. The facility may further include a system 80 configured to assess the gas composition measurements (e.g., to determine a confidence index that infers closeness of the gas measurements to the reservoir composition). The system may include the instrumentation or may be configured to receive the gas composition measurements from the instrumentation (e.g., the gas chromatography apparatus). The testing facility 60 may, of course, include numerous other testing instruments known to those of ordinary skill as well as computer hardware and software configured to assess the gas composition measurements and various drilling and environmental conditions. The hardware may include one or more processors (e.g., microprocessors) which may be connected to one or more data storage devices (e.g., hard drives or solid state memory) and user interfaces. It will be further understood that the disclosed embodiments may include processor executable instructions stored in the data storage device. The disclosed embodiments are, of course, not limited to the use of or the configuration of any particular computer hardware and/or software.

It will of course be appreciated that while FIG. 1 depicts a land rig 20, that the disclosed embodiments are equally well suited for land rigs or offshore rigs. As is known to those of ordinary skill, offshore rigs commonly include a platform deployed atop a riser that extends from the sea floor to the surface. The drill string extends downward from the platform, through the riser, and into the wellbore through a blowout preventer (BOP) located on the sea floor. The disclosed embodiments are expressly not limited in these regards.

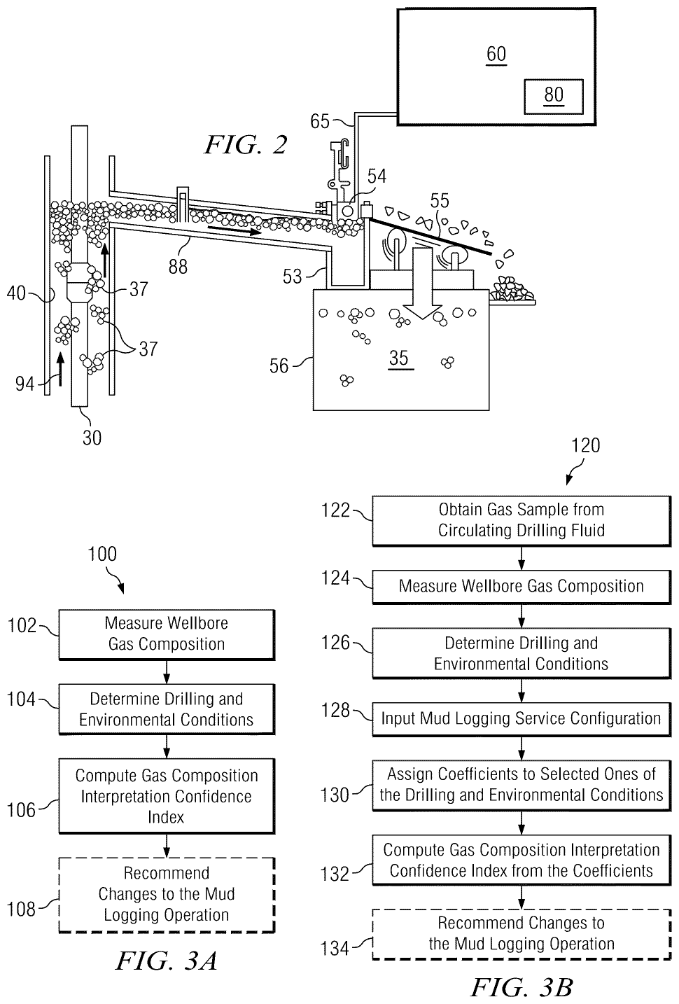

FIG. 2 depicts another view of a portion of surface system 50. As described above, the return conduit 52 is configured to carry drilling fluid 35 (sometimes including gas bubbles 37) from wellbore 40 to mud pit 56. The example system 50 includes a degasser 54 deployed, for example, in or near a header tank 53 that is immediately upstream of the shale shaker 55 and mud pit 56. In this example configuration, the degasser 54 is configured to remove gases in the drilling fluid that emerges from the wellbore 40 (referred to in the industry as gas-out). It will be appreciated that the disclosed embodiments are not limited in this regard. For example, the degasser 54 may include first and second degassers, the first configured to make gas-out measurements and the second deployed downstream of the mud pump so as to make gas-in measurements.

It will be further appreciated that the disclosed embodiments are not limited to the use of a degasser as depicted. Alternative embodiments may also (or additionally) make use of a gas probe located in the conduit 52 or at the surface of the well 40. In example embodiments, the degasser (or degassers) 54 may be piped directly to the mud logging unit or rig laboratory 60 (e.g. as depicted at 65), for example, to automatically transport the sampled gases for compositional testing.

It will be appreciated that system 50 may include substantially any suitable degasser (or degassers) 54, for example, including a vacuum degasser, a centrifugal degasser, and an impeller degasser. The degasser 54 may further be configured to heat the drilling fluid 35 to promote enhanced degassing of the fluid. The disclosed embodiments are not limited in regard to the type of degasser employed. Moreover, while not depicted, the system 50 may include one or more pumps (e.g., suction or pressure boosting pumps) configured to pump sampled gas from the degasser(s) 54 and/or the gas probe to the laboratory 60. The disclosed embodiments are, of course, not limited in regards to any sampling, pumping, or gas transport configurations.

FIG. 3A and 3B (collectively FIG. 3) depict flow charts of example methods 100 and 120 for making and assessing gas composition measurements. Methods 100 and 120 may be conducted, for example, while drilling a subterranean wellbore. In FIG. 3A, concentrations of selected alkane gases in circulating drilling fluid may be measured at 102 while drilling the wellbore. Various drilling and environmental conditions, such as weight on bit (WOB), drill string rotation rate (RPM), rate of penetration (ROP), atmospheric/weather conditions including temperature and atmospheric pressure, and drilling fluid temperature may be measured/determined at 104 while drilling the wellbore. The determined conditions may be evaluated at 106 along with a designated mud logging service configuration (e.g., including the drilling fluid degassing configuration and the measurement apparatus) to compute a gas composition interpretation confidence index (ICI). The computed ICI may provide an indication of how close the gas composition measurements made at 102 are to the actual reservoir fluid (gas) composition (or how confidently the measurements may be trusted to indicate the actual reservoir composition and properties). Method 100 may further, optionally, include evaluating the computed ICI, the measured conditions, and the known configuration of the drilling rig surface system and drilling fluid degassing configuration to recommend changes to the mud logging operation (e.g., a new service configuration) to improve the ICI at 108.

In FIG. 3B, method 120 may include obtaining gas samples via degassing drilling fluid used while drilling a wellbore at 122, measuring concentrations of selected alkane gases in the gas samples at 124, and monitoring/measuring the above described various drilling and environmental conditions at 126 while drilling the well. The degassing/measurement configuration (the service configuration) may be defined (input) at 128, for example, by an operator. Coefficients may be assigned to each of selected ones of the drilling and environmental conditions at 130, for example, based on the defined gas extraction/measurement configuration at 128 and the measured values of the drilling conditions at 126. A gas composition interpretation confidence index (ICI) may be computed at 132 from the assigned coefficients (e.g., by computing a sum or weighted sum of the assigned coefficients based upon imposed criteria). As described above with respect to method 100, the computed ICI may provide an indication of how close the gas composition measurements made at 122 are to the actual reservoir fluid (gas) composition (as well as how close the interpreted fluid type and properties are to the actual fluid type and properties). Method 120 may further, optionally, include evaluating the coefficients at 134 to recommend changes to the drilling and mud logging operations to improve the ICI.

With continued reference to FIG. 3, the gas measurements may be made at 102 and 124 while drilling (e.g. at substantially any suitable time interval while drilling the well) and the measured composition (e.g., the molecular composition) may be matched with the depth from which the hydrocarbon originated during drilling using mud logging techniques known to those of ordinary skill. Moreover, the measurements may be made using substantially any suitable service configuration including substantially any gas measurement apparatus and/or degasser. Moreover, in certain embodiments, a rig may include first and second mud logging systems, for example, a first utilized primarily for safety of the drilling operation and a second for evaluating the reservoir. In such embodiments, an ICI may be computed for measurements made by either or both of the of the systems.

The gas measurements may be made, for example, using a gas chromatography (GC) apparatus and/or a mass spectrometer (MS) apparatus and may quantify the amounts of light alkanes and optionally other gases (e.g., alkenes and alcohols) in the gas sample. In preferred embodiments, the gas measurements quantify at least the following light alkane gases: methane (C1), ethane (C2), propane (C3), n-butane (nC4), iso-butane (iC4), n-pentane (nC5), and iso-pentane (iC5), however, the disclosed embodiments are not limited in this regard. In other embodiments, the gas measurement apparatus may quantify C1 through C8 as well as aromatics such as benzene and toluene, cyclics such as cyclopentane, methylcyclohexane and inorganics such as hydrogen, helium, carbon monoxide, and carbon dioxide.

The gas samples may be obtained using substantially any suitable degasser configuration, for example, including a vacuum degasser, a centrifugal degasser, and an impeller degasser. The degasser may further be configured to heat the drilling fluid to promote full degassing of the fluid. Moreover, the gas sample obtained via degassing the drilling fluid may be pumped (e.g., via a suction or pressure boosting pumps) to the measurement apparatus in the laboratory (as described above with respect to FIG. 2).

In certain embodiments, the service configuration may include, for example, the Fluid Logging Analysis in Real Time (“FLAIR”) and FlairFlex service, offered by SLB. FLAIR FlairFlex are quantitative mud logging services that provides lab quality gas composition measurements of the reservoir fluids during drilling and makes use of GC-MS to measure C1 through C5. FLAIR and FlairFlex-like data may be used for hydrocarbon and fluid contacts identification and inter- and intra-well fluid facies mapping and is described herein as an example surface fluid logging service.

It will be appreciated that the disclosed embodiments are not limited to the use of FLAIR or FlairFlex and that other gas analyzers may be used to obtain gas composition measurements that may be assessed using the disclosed embodiments. The service configuration may include substantially any other suitable gas measurement apparatus and degasser (commercial or not yet commercial). In other embodiments, the service configuration may alternatively include various ones of the PureFlex mud logging service configurations (available from SLB) or other reservoir fluid analysis (service) configurations. The gas measurement apparatus may include, for example, GC-FID, GC-TCD, GC-MS, MS, adsorption spectroscopy (e.g., IR, MID), and/or Raman spectroscopy.

With continued reference to FIG. 3, the drilling and environmental conditions measured at 104 and 126 may include substantially any suitable conditions. For example, the conditions may include the type of drilling fluid in use in the well, for example, oil-based mud (OBM) or water-based mud (WBM), the temperature of the drilling fluid, the drilling WOB, and the drilling RPM, the drilling rate of penetration (ROP), as well as various atmospheric/weather conditions including temperature and atmospheric pressure as well as day night temperature variations. The measured conditions may further include an evaluation of drilling fluid contamination and an evaluation of biodegradation of the obtained gas sample. Drilling fluid contamination may be evaluated, for example, by computing a ratio of butane and/or pentane to propane (e.g., C4/C3, C5/C3, or C4·C5/C3) in the gas composition measurements. Biodegradation may be evaluated, for example, by computing a ratio of iso-butane to n-butane (iC4/nC4), iso-pentane to n-pentane (iC5/nC5), or ethane to propane (C2/C3) in the gas composition measurements. The type of extraction efficiency calibration may also be considered.

The service configuration may be defined according to any criteria. For example, the make and type of degasser may be defined, for example, a heated or non-heated degasser. Moreover, the number of employed degassers may be defined, for example, including a single gas-out degasser or a first gas-out degasser and a second gas-in degasser. The length of the gas line between the degasser(s) and the measurement apparatus may also be defined. The type of gas measurement apparatus may also be defined, for example, thereby defining the range of alkane gases measured (e.g., C1-C5 or C1-C8) as well as other gases that may be measured (e.g., alkenes or alcohols). Additional services (such as isotope measurement) and features (such as mud contamination removal) may also be defined.

Selected ones of the drilling and environmental conditions may be evaluated in view of the defined service configuration to assign the aforementioned coefficients at 130. In example embodiments, the coefficient may represent a penalty such as a negative penalty for a non-optimal condition. Conditions that are less optimal (more nonoptimal) may result in a larger penalty (a more highly negative coefficient). Moreover, the coefficient may be set to zero when the conditions are taken to be optimal or near optimal (such that no penalty is applied).

For example, a coefficient may be assigned based on the drilling fluid surface temperature or the temperature of the drilling fluid arriving at the degasser. In such embodiments, the criteria may be based upon a measured surface temperature. For a service configuration employing a non-heated degasser, the coefficient (penalty) may be highly negative when the measured temperature of the drilling fluid arriving at the degasser is low (e.g., a temperature less than a threshold). The coefficient may be less negative at intermediate temperatures and zero (best case) when the surface temperature of the drilling fluid is high (above a threshold). The increasing efficiency of gas extraction with increasing fluid temperature may provide the rationale for this particular coefficient. In other example embodiments, when the service configuration employees a heated degasser, the coefficient may be independent of temperature and may be set to zero indicating no penalty.

As drilling progresses in depth, formations generally get hotter. As a result the deeper the well, the hotter the mud is expected to return to the surface (pending other factors such geothermal gradient, mud flow rate, mud cooling at the surface, and heat of mud dissipation into the rocks of the borehole the mud is travelling through via the well's annulus). Hence, the first drilled meters of a well or section may provide the first return of mud to the surface and a reference temperature that may be recorded. As drilling progresses subsequent mud temperature readings may be compared to the reference. When a non-heated degasser is used increasing penalties may be applied for higher differences.

In still other example embodiments a large penalty may be applied (highly negative coefficient) when the drilling fluid is an oil-based mud (OBM) and a comparatively smaller penalty may be applied (less negative coefficient) when the drilling fluid is a water-based mud (WBM). Moreover, this penalty may only be applied for certain service configurations. In other service configurations, the penalty may only be applied when OBM is used. And in still other service configurations no penalty is applied irrespective of the type of drilling fluid employed.

In example embodiments, the coefficient may indicate conditions at which drill bit metamorphism (DBM) is likely. In such embodiments, the coefficient may depend upon whether OBM or WBM is employed in the wellbore. For example, a larger penalty (more negative coefficient) may be assigned when OBM is employed. Moreover, the coefficients may depend upon the measured ROP and/or RPM. For example, a first DBM coefficient may be assigned based upon the ROP and a second DBM coefficient may be assigned based upon the RPM. In such embodiments, the coefficients may be more highly negative when the ROP is low (below a threshold) and when the RPM is high (above a threshold). The coefficients may be set to zero when the ROP is above a threshold and the RPM is below a threshold. Intermediate coefficient values may also be assigned.

In still other example embodiments, a condensation coefficient may be assigned. For example, the condensation coefficient may be related to the temperature that the gas line is exposed to, such as a difference between day and nighttime temperatures and the length of the gas line. In service configurations that employ a non-heated gas line, cold temperatures may cause condensation of C4, C5, and above, particularly in a long gas line (thereby influencing measured gas ratios and the corresponding interpretation). A large penalty (highly negative coefficient) may be applied when the difference between the day and night temperatures exceeds a threshold or when both day and night temperatures are below a threshold. A smaller penalty (or no penalty) may be applied when the difference between the day and night temperatures is small (less than a threshold) or when both day and night temperatures are warm (above a threshold). Intermediate coefficients may also be applied. Moreover, in example embodiments, the condensation coefficient may only be applied when a nonheated gas line is used.

A biodegradation coefficient may also be assigned. For example, a highly negative coefficient may be assigned when a ratio between iC4 and nC4 or between iC5 and nC5 exceeds a threshold. A less negative coefficient may be assigned at smaller, intermediate ratios and no penalty may be assigned when the ratio is less than a threshold.

A mud contamination coefficient that is intended to identify contamination of the drilling fluid with polar components (such as alcohols, amines, and ammonia), particularly in WBM, may also be assigned. For example, a highly negative coefficient may be assigned when a ratio of C4C5 to C3 exceeds a threshold. A less negative coefficient may be assigned at smaller, intermediate ratios and no penalty may be assigned when the ratio is less than a threshold.

An extraction efficiency coefficient (EEC) may also be assigned, based on the pre-drilling experimental assignment of an EEC to each of the measured alkanes or by statistical assignment of arbitrary values. The former method allows quantitative real-time gas-in-mud measurement, and may obtain no penalty (e.g., like in FlairFlex option with EEC-calibration mud service), while the latter may obtain a small penalty. Lack of any EEC (which is possible only with a heated degasser) may result in maximum penalty. Moreover, in some example embodiments, the extraction efficiency coefficient may be positive, indicating a benefit to employing such techniques. For example, the use of certain techniques may provide a small positive coefficient while others may provide a large positive coefficient. Still others may provide no benefit such that the coefficient may be set to zero.

It will be appreciated that in certain example embodiments the coefficients may be assigned automatically with the use of a lookup table. For example, a suitable lookup table may include a listing of tabulated coefficients for each of the selected environmental and measurement conditions at each of the potential service configurations. In such embodiments, the service configuration may be preselected (e.g., manually) at the beginning of the mud logging operation (e.g., by selecting the type of degasser and measurement apparatus utilized). The mud type, temperature sensor types, and extraction efficiency calibration may also be preselected.

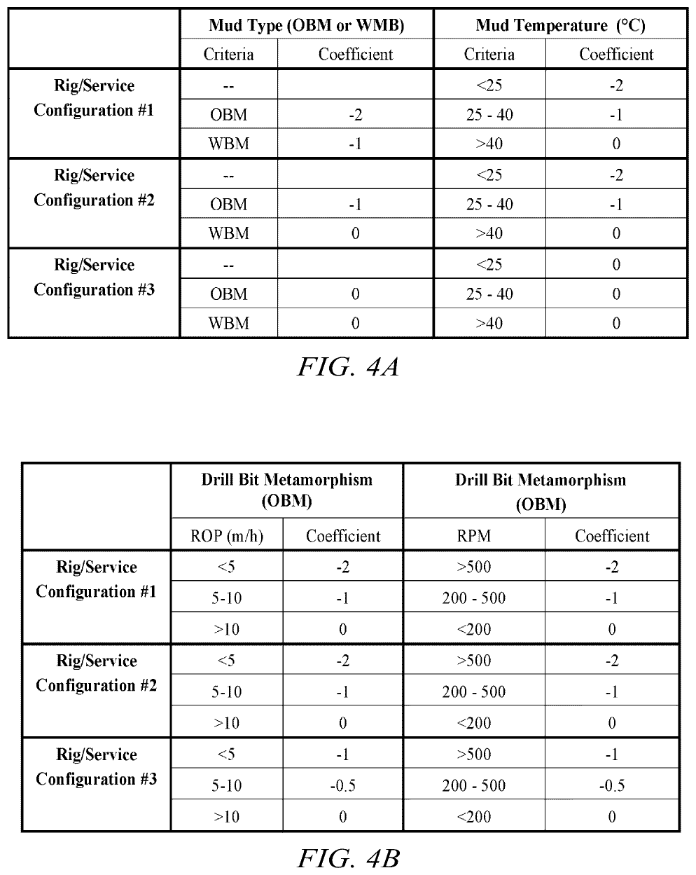

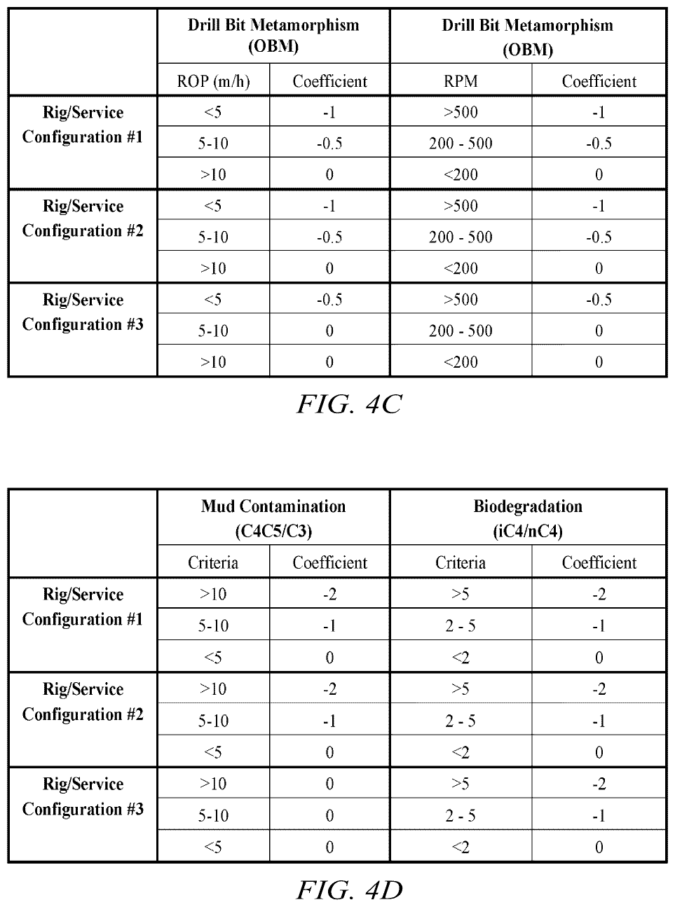

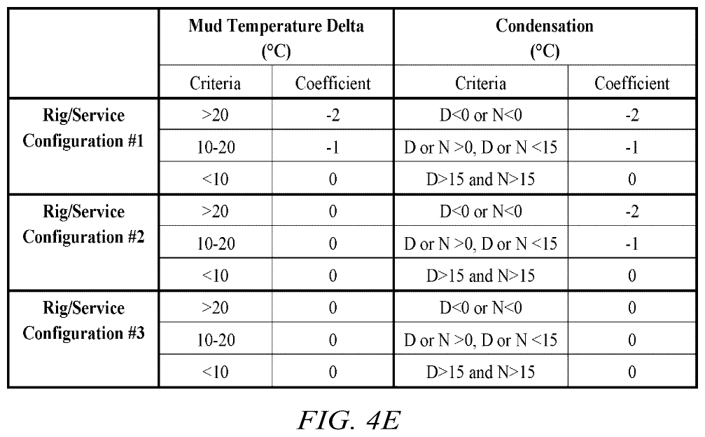

Turning now to FIGS. 4A, 4B, 4C, 4D, and 4E (collectively FIG. 4), the disclosed embodiments are described in more detail with respect to the following non-limiting example. In this example, coefficients are listed for different environmental and measurement conditions for three distinct example service configurations. It will be appreciated that the disclosed embodiments are in no way limited by the criteria and coefficients depicted herein. Nor are the disclosed embodiments limited to any particular environmental and measurement conditions or to any number of distinct service configurations. It will be appreciated that, in practice, a large number of potential service configurations may be available and that additional environmental and measurement conditions may be employed. It will be further appreciated that the coefficient values may further depend on the nature of the field in which the wellbore is drilled (e.g., characteristics of the reservoir and its contents).

In FIG. 4A, example mud type and mud temperature coefficients are listed for three example service configurations. In this particular example, mud type coefficients of −2 and −1 may be assigned when using OBM and WBM with the first service configuration. For the second service configuration a mud type coefficient of −1 may be assigned when using OBM and a mud type coefficient of 0 may be assigned when using WBM. For the third service configuration a mud type coefficient of 0 may be assigned for either OBM or WBM. Mud temperature coefficients of −2 and −1 may be assigned when the mud temperature is less than 25° C. and between 25° C. and 40° C. for each of the first and second service configurations. The mud temperature coefficient may be set to zero when the temperature is greater than 40° C. The mud temperature coefficient may also be set to zero at all mud temperatures when using the third service configuration (which may include, for example, a heated degasser).

In FIG. 4B, example first and second DBM coefficients are listed for three example service configurations when using OBM. In this particular example, a first DBM coefficient of −2 and −1 may be assigned when the ROP is less than 5 m/h and between 5 and 10 m/h for each of the first and second service configurations. The first DBM coefficient may be set to zero when the ROP is greater than 10 m/h. For the third service configuration, the first DBM coefficient may be half that of the first and second service configurations. Moreover, a second DBM coefficient of −2 and −1 may be assigned when the RPM is greater than 500 and between 200 and 500 for each of the first and second service configurations. The second DBM coefficient may be set to zero when the RPM is less than 200. For the third service configuration, the second DBM coefficient may be half that of the first and second service configurations.

In FIG. 4C, example first and second DBM coefficients are listed for three example service configurations when using WBM. In this particular example, first and second DBM coefficients may be half of that assigned when using OBM (FIG. 4B) for each of the first and second service configurations. For the third service configuration the first DBM coefficient may be set to 0.5 when the ROP is less than 5 m/h and the second DBM coefficient may be set to 0.5 when the RPM is greater than 500. Otherwise, the first and second DBM coefficients may be set to zero.

In FIG. 4D, example mud contamination and biodegradation coefficients are listed for three example service configurations. In this particular example, for the first and second service configurations a mud contamination coefficient may be assigned a value of −2 when a ratio of C4C5 to C3 exceeds 10, a value of −1 when the ratio is between 5 and 10, and a value of 0 when the ratio is less than 5. For the third service configuration, the mud contamination coefficient may be set to zero for all ratios. As also shown, for each of the service configurations a biodegradation coefficient may be assigned a value of −2 when a ratio of iC4 nC4 exceeds 5, a value of −1 when the ratio is between 2 and 5, and a value of 0 when the ratio is less than 5.

In FIG. 4E, example mud temperature delta and condensation coefficients are listed for three example service configurations. It will be appreciated that mud temperature delta is a measure of the difference between the measured surface drilling fluid temperature and a reference temperature measurement of the surface drilling fluid temperature at the beginning of the well or well section. In this particular example, the mud temperature delta coefficient may be set to −2 when the temperature difference is greater than 20° C., −1 when the temperature difference between 10° C. and 20° C., and 0 when the temperature difference is less than 10° C. for the first service configuration. The mud temperature delta coefficient may be set to zero for all measured temperature differences for the for the second and third service configurations.

Moreover, for the first two service configurations, the condensation coefficient may be set to −2 when the day temperature and/or the night temperature is below 0° C., −1 when the day and night temperature are both above 0° C. and at least one of the day and night temperature are below 15° C., and 0 when the day temperature and the night temperature are both above 15° C. The condensation coefficient may be set to zero for all day and night temperatures for the for the third service configuration.

With continued reference to FIG. 3, the confidence index may be computed at substantially any suitable interval while drilling the well (e.g., whenever gas measurements are made). The computed confidence index may also be displayed in a log, for example, depicting the confidence index value as a function of measured depth of the wellbore. The confidence index may be depicted in the log as a pseudo-color indicator or as a line graph. The disclosed embodiments are not limited in this regard.

As further described above with respect to FIG. 3, the confidence index may be evaluated to provide recommendations. For example, the environmental and measurement conditions having large negative coefficients (penalties) may be identified and recommendations may be made to reduce these coefficients and therefore improve the confidence index.

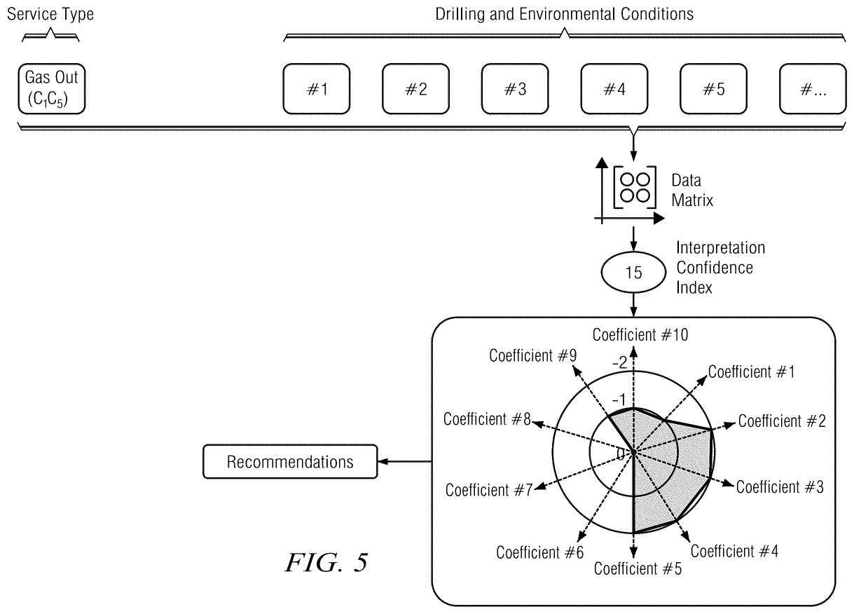

FIG. 5 depicts a graphical representation of coefficients for a plurality of example environmental and measurement conditions for a given depth interval. In the depicted example, the assigned coefficients for each of ten environmental and measurement conditions are depicted along corresponding axes. In this particular example, coefficients 2, 3, 4, and 5 may be readily identified as being highly negative (−2 in this particular example), while coefficients 1, 9, and 10 may be identified as being moderately negative (−1 in this particular example). To improve the confidence index (i.e., to achieve a confidence index closer to zero), recommendations may be made, for example, to modify the service configuration such that coefficients 2, 3, 4, and 5 are less negative (e.g., have a value of 0 or −1 in this particular example).

It will be understood that the present disclosure includes numerous embodiments. These embodiments include, but are not limited to, the following embodiments.

In a first embodiment, a method for assessing downhole gas composition measurements is disclosed. The method comprises measuring a composition of a gas sample obtained during a mud logging operation, the composition including at least selected alkane gases; determining drilling and environmental conditions corresponding to the measuring a composition; and computing an interpretation confidence index for the measured composition from selected ones of the drilling and environmental conditions, wherein the interpretation confidence index provides an assessment of the how well the measured composition reflects an actual reservoir gas composition.

A second embodiment may include the first embodiment, further comprising recommending a change to the mud logging operation based on the computed interpretation confidence index.

A third embodiment may include any one of the first through second embodiments, wherein the measuring the composition comprises using a gas chromatography apparatus or a mass spectrometer to measure the composition.

A fourth embodiment may include any one of the first through third embodiments, further comprising designating a mud logging service configuration including a type of degasser used to obtain the gas sample and a type of gas measurement apparatus used to make the gas composition measurement, wherein the evaluating further comprises evaluating the drilling and environmental conditions and the designated mud logging service configuration to estimate the interpretation confidence index.

A fifth embodiment may include any one of the first through fourth embodiments, wherein the measuring the composition, further comprises degassing a portion of the circulating drilling fluid to obtain the gas sample; and measuring the composition of the obtained gas sample.

A sixth embodiment may include any one of the first through fifth embodiments, wherein the evaluating the drilling and environmental conditions further comprises assigning a coefficient for each of selected ones of the determined drilling and environmental conditions; and summing the assigned coefficients to compute the interpretation confidence index.

A seventh embodiment may include the sixth embodiment, wherein the coefficient comprises first and second drill bit metamorphism (DBM) coefficients; the first DBM coefficient assigned based on a measured rate of penetration of drilling and a type of drilling fluid from which the gas sample was obtained; and the second DBM coefficient assigned based on a measured rotation rate of a drill bit while drilling and a type of drilling fluid from which the gas sample was obtained.

An eighth embodiment may include any one of the sixth through seventh embodiments, wherein the coefficient comprises a drilling fluid contamination coefficient and a biodegradation coefficient; the drilling fluid contamination coefficient is assigned based on a ratio of butane and/or pentane to propane in the measured composition; and the biodegradation coefficient is assigned based on a ratio iso butane to normal butane or iso pentane to normal pentane in the measured composition.

A ninth embodiment may include any one of the sixth through eighth embodiments, wherein the coefficient comprises a mud temperature coefficient and a condensation coefficient; the mud temperature coefficient is assigned based on a measured surface temperature of the drilling fluid; and the condensation coefficient is assigned based on day and night temperature measurements made at a rig site where the mud logging operation is performed.

A tenth embodiment may include any one of the sixth through ninth embodiments, wherein the assigning the coefficients and the summing the assigned coefficients is performed automatically for each gas composition measurement.

In an eleventh embodiment a system for assessing downhole gas composition measurements is disclosed. The system comprises a measurement apparatus configured to measure a composition of a gas sample obtained during a mud logging operation, the composition including at least selected alkane gases; and a processor configured to receive a measured gas composition from the measurement apparatus; receive drilling and environmental conditions corresponding to the measuring a composition; and evaluate the drilling and environmental conditions to estimate an interpretation confidence index for the measured composition, wherein the interpretation confidence index provides an assessment of the how well the measured composition reflects an actual reservoir gas composition.

A twelfth embodiment may include the eleventh embodiment, wherein the processor is further configured to receive a designated a mud logging service configuration including a type of degasser used to obtain the gas sample and a type of gas measurement apparatus used to make the gas composition measurement and to evaluate the drilling and environmental conditions and the designated mud logging service configuration to estimate the interpretation confidence index.

A thirteenth embodiment may include the twelfth embodiment, wherein the processor is further configured to assign a coefficient for each of selected ones of the received drilling and environmental conditions; and sum the assigned coefficients to compute the interpretation confidence index.

A fourteenth embodiment may include the thirteenth embodiment, further comprising a lookup table stored in electronic memory, the lookup table including a listing of coefficients for each of the selected environmental and drilling conditions for a plurality of distinct mud logging service configurations, wherein the processor is configured to retrieve the assigned coefficient from the lookup table based on the selected environmental and drilling conditions and the designated mud logging service configuration.

A fifteenth embodiment may include the fourteenth embodiment, wherein the lookup table comprises at least the following coefficients a first drill bit metamorphism (DBM) coefficient that is assigned based on a measured rate of penetration of drilling and a type of drilling fluid from which the gas sample was obtained; a second DBM coefficient that is assigned based on a measured rotation rate of a drill bit while drilling and a type of drilling fluid from which the gas sample was obtained; a drilling fluid contamination coefficient that is assigned based on a ratio of butane and/or pentane to propane in the measured composition; a biodegradation coefficient that is assigned based on a ratio iso butane to normal butane or iso pentane to normal pentane in the measured composition; a mud temperature coefficient that is assigned based on a measured surface temperature of the drilling fluid; and a condensation coefficient that is assigned based on day and night temperature measurements made at a rig site where the mud logging operation is performed.

In a sixteenth embodiment a method for assessing downhole gas composition measurements is disclosed. The method comprises obtaining a gas sample from circulating drilling fluid during a mud logging operation; measuring a composition the gas sample, the composition including at least selected alkane gases; determining drilling and environmental conditions corresponding to the measuring a composition; designating a mud logging service configuration including a type of degasser used to obtain the gas sample and a type of gas measurement apparatus used to make the gas composition measurement; assigning coefficients to selected ones of the determined drilling and environmental conditions based upon the designated mud logging service configuration; computing an interpretation confidence from the assigned coefficients, wherein the interpretation confidence index provides an assessment of the how well the measured composition reflects an actual reservoir gas composition.

A seventeenth embodiment may include the sixteenth embodiment, further comprising recommending a change to the mud logging operation based on the assigned coefficients and the computed interpretation confidence index.

An eighteenth embodiment may include any one of the sixteenth through seventeenth embodiments, wherein the assigned coefficients comprise at least the following coefficients a first drill bit metamorphism (DBM) coefficient that is assigned based on a measured rate of penetration of drilling and a type of drilling fluid from which the gas sample was obtained; a second DBM coefficient that is assigned based on a measured rotation rate of a drill bit while drilling and a type of drilling fluid from which the gas sample was obtained; a drilling fluid contamination coefficient that is assigned based on a ratio of butane and/or pentane to propane in the measured composition; a biodegradation coefficient that is assigned based on a ratio iso butane to normal butane or iso pentane to normal pentane in the measured composition; a mud temperature coefficient that is assigned based on a measured surface temperature of the drilling fluid; and a condensation coefficient that is assigned based on day and night temperature measurements made at a rig site where the mud logging operation is performed.

A nineteenth embodiment may include any one of the sixteenth through eighteenth embodiments, wherein the computing comprises summing the assigned coefficients to compute the interpretation confidence index.

A twentieth embodiment may include any one of the sixteenth through nineteenth embodiments, wherein the assigning coefficients further comprises retrieving the assigned coefficient from a lookup table based on the selected environmental and drilling conditions and the designated mud logging service configuration, wherein the lookup table includes a listing of coefficients for each of the selected environmental and drilling conditions for a plurality of distinct mud logging service configurations.

Although methods for evaluating wellbore gas measurements have been described in detail, it should be understood that various changes, substitutions and alternations can be made herein without departing from the spirit and scope of the disclosure as defined by the appended claims.

What is claimed is:

1. A method for assessing downhole gas composition measurements, the method comprising:
measuring a composition of a gas sample obtained during a mud logging operation, the composition including at least selected alkane gases;
determining drilling and environmental conditions corresponding to the measuring a composition; and
computing an interpretation confidence index for the measured composition from selected ones of the drilling and environmental conditions, wherein the interpretation confidence index provides an assessment of the how well the measured composition reflects an actual reservoir gas composition.

2. The method of claim 1, further comprising recommending a change to the mud logging operation based on the computed interpretation confidence index.

3. The method of claim 1, wherein the measuring the composition comprises using a gas chromatography apparatus or a mass spectrometer to measure the composition.

4. The method of claim 1, further comprising designating a mud logging service configuration including a type of degasser used to obtain the gas sample and a type of gas measurement apparatus used to make the gas composition measurement, wherein the evaluating further comprises evaluating the drilling and environmental conditions and the designated mud logging service configuration to estimate the interpretation confidence index.

5. The method of claim 1, wherein the measuring the composition, further comprises:
degassing a portion of the circulating drilling fluid to obtain the gas sample; and
measuring the composition of the obtained gas sample.

6. The method of claim 1, wherein the evaluating the drilling and environmental conditions further comprises:
assigning a coefficient for each of selected ones of the determined drilling and environmental conditions; and
summing the assigned coefficients to compute the interpretation confidence index.

7. The method of claim 6, wherein:
the coefficient comprises first and second drill bit metamorphism (DBM) coefficients;
the first DBM coefficient assigned based on a measured rate of penetration of drilling and a type of drilling fluid from which the gas sample was obtained; and
the second DBM coefficient assigned based on a measured rotation rate of a drill bit while drilling and a type of drilling fluid from which the gas sample was obtained.

8. The method of claim 6, wherein:
the coefficient comprises a drilling fluid contamination coefficient and a biodegradation coefficient;
the drilling fluid contamination coefficient is assigned based on a ratio of butane and/or pentane to propane in the measured composition; and
the biodegradation coefficient is assigned based on a ratio iso butane to normal butane or iso pentane to normal pentane in the measured composition.

9. The method of claim 6, wherein:
the coefficient comprises a mud temperature coefficient and a condensation coefficient;
the mud temperature coefficient is assigned based on a measured surface temperature of the drilling fluid; and
the condensation coefficient is assigned based on day and night temperature measurements made at a rig site where the mud logging operation is performed.

10. The method of claim 6, wherein the assigning the coefficients and the summing the assigned coefficients is performed automatically for each gas composition measurement.

11. A system for assessing downhole gas composition measurements, the system comprising:
a measurement apparatus configured to measure a composition of a gas sample obtained during a mud logging operation, the composition including at least selected alkane gases; and
a processor configured to:
receive a measured gas composition from the measurement apparatus;
receive drilling and environmental conditions corresponding to the measuring a composition; and
evaluate the drilling and environmental conditions to estimate an interpretation confidence index for the measured composition, wherein the interpretation confidence index provides an assessment of the how well the measured composition reflects an actual reservoir gas composition.

12. The system of claim 11, wherein the processor is further configured to receive a designated a mud logging service configuration including a type of degasser used to obtain the gas sample and a type of gas measurement apparatus used to make the gas composition measurement and to evaluate the drilling and environmental conditions and the designated mud logging service configuration to estimate the interpretation confidence index.

13. The system of claim 12, wherein the processor is further configured to:
assign a coefficient for each of selected ones of the received drilling and environmental conditions; and
sum the assigned coefficients to compute the interpretation confidence index.

14. The system of claim 13, further comprising a lookup table stored in electronic memory, the lookup table including a listing of coefficients for each of the selected environmental and drilling conditions for a plurality of distinct mud logging service configurations, wherein the processor is configured to retrieve the assigned coefficient from the lookup table based on the selected environmental and drilling conditions and the designated mud logging service configuration.

15. The system of claim 14, wherein the lookup table comprises at least the following coefficients:
a first drill bit metamorphism (DBM) coefficient that is assigned based on a measured rate of penetration of drilling and a type of drilling fluid from which the gas sample was obtained;
a second DBM coefficient that is assigned based on a measured rotation rate of a drill bit while drilling and a type of drilling fluid from which the gas sample was obtained;
a drilling fluid contamination coefficient that is assigned based on a ratio of butane and/or pentane to propane in the measured composition;
a biodegradation coefficient that is assigned based on a ratio iso butane to normal butane or iso pentane to normal pentane in the measured composition;
a mud temperature coefficient that is assigned based on a measured surface temperature of the drilling fluid; and
a condensation coefficient that is assigned based on day and night temperature measurements made at a rig site where the mud logging operation is performed.

16. A method for assessing downhole gas composition measurements, the method comprising:
obtaining a gas sample from circulating drilling fluid during a mud logging operation;
measuring a composition the gas sample, the composition including at least selected alkane gases;
determining drilling and environmental conditions corresponding to the measuring a composition;
designating a mud logging service configuration including a type of degasser used to obtain the gas sample and a type of gas measurement apparatus used to make the gas composition measurement;
assigning coefficients to selected ones of the determined drilling and environmental conditions based upon the designated mud logging service configuration;
computing an interpretation confidence from the assigned coefficients, wherein the interpretation confidence index provides an assessment of the how well the measured composition reflects an actual reservoir gas composition.

17. The method of claim 16, further comprising recommending a change to the mud logging operation based on the assigned coefficients and the computed interpretation confidence index.

18. The method of claim 16, wherein the assigned coefficients comprise at least the following coefficients:
a first drill bit metamorphism (DBM) coefficient that is assigned based on a measured rate of penetration of drilling and a type of drilling fluid from which the gas sample was obtained;
a second DBM coefficient that is assigned based on a measured rotation rate of a drill bit while drilling and a type of drilling fluid from which the gas sample was obtained;
a drilling fluid contamination coefficient that is assigned based on a ratio of butane and/or pentane to propane in the measured composition;
a biodegradation coefficient that is assigned based on a ratio iso butane to normal butane or iso pentane to normal pentane in the measured composition;
a mud temperature coefficient that is assigned based on a measured surface temperature of the drilling fluid; and
a condensation coefficient that is assigned based on day and night temperature measurements made at a rig site where the mud logging operation is performed.

19. The method of claim 16, wherein the computing comprises summing the assigned coefficients to compute the interpretation confidence index.

20. The method of claim 16, wherein the assigning coefficients further comprises retrieving the assigned coefficient from a lookup table based on the selected environmental and drilling conditions and the designated mud logging service configuration, wherein the lookup table includes a listing of coefficients for each of the selected environmental and drilling conditions for a plurality of distinct mud logging service configurations.
